(19) United States
(12) Patent Application Publication
(10) Pub. No.: US 2026/0185957 A1
(43) Pub. Date: Jul. 2, 2026

(54) ELECTROCHEMICAL IMMUNOASSAY

(71) Applicant: Roche Diagnostics Operations, Inc., Indianapolis, IN (US)

(72) Inventors: Oscar GUTIÉRREZ-SANZ, Heidelberg (DE); Eloisa LOPEZ-CALLE, Ludwigshafen (DE); Hanna OSTERHELD, Walldorf (DE); Bhawana THAKUR, Mannheim (DE); Tabea TREU, Karlsruhe (DE)

(21) Appl. No.: 19/545,957

(22) Filed: Feb. 20, 2026

Related U.S. Application Data
(63) Continuation of application No. PCT/EP2024/073570, filed on Aug. 22, 2024.

(30) Foreign Application Priority Data
Aug. 23, 2023 (EP) 23192850.8

(51) Int. Cl. G01N 27/327; G01N 27/30; G01N 33/543; G01N 33/68

(52) U.S. Cl. CPC G01N 27/3277; G01N 27/301; G01N 33/54366; G01N 33/68; G01N 2333/58

(57) ABSTRACT

There is disclosed an electrode for an electrochemical immunoassay; wherein the electrode is functionalized with at least one antibody or fragment thereof configured to bind to at least one analyte; and wherein the at least one antibody or fragment thereof is conjugated with at least one redox species such that the electrochemical activity of the at least one redox species is altered when the at least one antibody or fragment thereof binds to the at least one analyte.

### CLAIM OF PRIORITY

This application is a continuation of PCT/EP 2024/073570 (filed on Aug. 22, 2024), which claims priority to and benefit of European Patent No. 23192850.8 (filed on Aug. 23, 2023). The contents of these applications are incorporated herein by reference in their entirety.

### TECHNICAL FIELD

Certain examples of the present disclosure relate to an electrode for an electrochemical immunoassay, a sensor comprising the electrode, and a method of monitoring an analyte concentration with the electrode or the sensor.

### BACKGROUND

Amongst a vast array of techniques, electrochemical immunoassay can provide a feasible pathway to develop robust, miniaturized, and low-cost sensors for analyte detection, especially for in-situ protein detection. Electrochemical immunoassays use electrochemical techniques to detect binding of analytes to antibodies or fragments thereof immobilised on an electrode surface. Such peptidic binding agents, however, lack appreciable redox activity, which makes their sensitive electrochemical detection challenging. To achieve desirable sensitivity, one of the conventional approaches is to use additional redox molecules in the sample matrix or to modify the electrode surface with the redox molecules. In such sensors, the antibody-antigen binding event is marked by a change in the electroactivity of the redox probe. Another approach for electrochemical immunoassay involves tagging of antibodies with enzymes and using an additional reagent i.e. enzyme specific substrate. Herein, the antibody-antigen recognition event is followed by generation of an electroactive species owing to the enzymatic action.

These existing electrochemical immunoassays are not wash-free and the need to add additional chemicals to the sample solutions makes them impractical for use in in-situ continuous monitoring applications, for example as wearable or implantable sensors.

In other types of biosensors, a redox label is attached to oligonucleotides such as DNA aptamers (oligo-redox). The oligo-redox is anchored on the surface of sensor. Upon binding with the biomarker, the Oligo-redox undergoes change in its structure. This movement causes a change in the position of the redox species relative to the surface of sensor electrode.

However, the low stability of oligo-redox complexes in the presence of nucleases (often present in physiological samples) might challenge the performance of the sensors, especially in case multiple use of the sensors or continuous sensing is desired. Aptamers also have limited functional diversity, making their conjugation with molecules of interest (redox tags, enzyme tags, nanoparticles, etc.) challenging. Finally, the oligonucleotide based structure might limit the interactions of the oligo-redox complex with the target analyte.

The above information is presented as background information only to assist with an understanding of the present disclosure. No determination has been made, and no assertion is made, as to whether any of the above might be applicable as prior art with regard to the present invention.

### SUMMARY

It is an aim of certain examples of the present disclosure to address, solve and/or mitigate, at least partly, at least one of the problems and/or disadvantages associated with the related art, for example at least one of the problems and/or disadvantages described herein.

It is an aim of certain examples of the present disclosure to provide electrochemical immunoassay electrodes and sensors for detecting analytes in bodily fluids.

An electrode for an electrochemical immunoassay is functionalized with at least one antibody or fragment thereof configured to bind to at least one analyte. The at least one antibody or fragment thereof is conjugated with at least one redox species such that the electrochemical activity of the at least one redox species is altered when the at least one antibody or fragment thereof binds to the at least one analyte. This change in the electrochemical activity of the at least one redox species is caused by the binding event between the analyte and the at least one antibody or fragment thereof and can therefore be used as a parameter to determine the presence and/or concentration of the analyte.

The redox activity of a redox molecule is dependent on an efficient electron transfer between the redox molecule and the electrode surface. This electron transfer may be perturbed by a change in the microenvironment of the redox molecule. For the electrode described above, such a change may be caused when the antibody/fragment binds to the corresponding analyte, for example the change may be caused by the near-presence of the analyte and/or the conformational change of the antibody/fragment upon binding to the analyte.

That is, when the antibody/fragment binds to the analyte, the electrochemical activity of the redox species changes. Consequently, the presence/concentration of an analyte in a solution can be detected via a change of the electrochemical activity of the redox species when the working electrode is exposed to a solution. The change of the electrochemical activity of the redox species can be determined using an appropriate electrochemical read-out technique like voltammetry.

By using antibodies or fragments thereof conjugated to redox molecules, detection of the analyte can be performed without the need for additional chemicals. This reduces the problem that such additional and potentially harmful chemicals not fixed to the electrode can be released to the exposed solution in many alternative assay formats that make these alternative assay formats vastly inapplicable for in-situ applications. The use of antibodies or fragments thereof also provides enhanced selectivity.

Optionally, the at least one antibody or fragment thereof is conjugated with the at least one redox species via covalent bonding.

This may ensure a strong bond between the antibody/fragment and the redox species, and may reduce degradation of the sensor. Further, by using covalent bonding between the antibody/fragment and the redox species, the position of the conjugated redox mediators relative to the antibody/fragment remains stable and fixed over time and may allow reproducible successive analyte determinations and thereby a use of such electrodes in continuous monitoring applications.

Optionally, the at least one antibody or fragment thereof is bound to the electrode via covalent bonding, or the at least one antibody or fragment thereof is bound to the electrode via a streptavidin-biotin complex.

This may ensure a strong bond between the antibody/fragment and the electrode surface, and may further reduce degradation of the sensor.

Optionally, the electrode is functionalized with at least one antibody fragment comprising a F(ab′)2 fragment, a Fab fragment, a Fab′ fragment, an (sc)Fv-fragment, or a SMIP (small modular immunopharmaceutical).

By using an antibody fragment the redox species and/or the analyte binding site of the antibody may be closer to the electrode surface than if a complete antibody is used. Consequently, perturbations in the redox activity caused by changes in the microenvironment when the analyte binds to the antibody fragment may be more easily and sensitively detected, due to the reduced distance between the redox species conjugated to the antibody fragment and the electrode surface.

Optionally, the at least one redox species has a low oxidation potential. Preferably, the oxidation potential is 0.3 V or less. More preferably, the oxidation potential is 0.12 V or less.

An advantage of using a redox species with a low oxidation potential is that detection of the analyte can be performed at lower potentials to exclude or at least reduce interference effects caused by other redox active substances with higher oxidation potentials that may also be present in the sample. That is, using the lower oxidation potential may reduce the probability that other redox species in the sample will undergo a redox reaction and produce signals that would overlap with the signal from the at least one redox species conjugated to the antibody/fragment.

Optionally, the at least one redox species is one of Prussian blue, methylene blue, or an osmium complex.

Optionally, the at least one analyte is a peptide or protein.

Optionally, the at least one analyte is a cardiac related biomarker.

Optionally, the at least one analyte is a natriuretic peptide.

Optionally, the least one analyte is NT-proBNP.

A sensor comprises the electrode described above, wherein the electrode described above is a working electrode of the sensor. The sensor further comprises an auxiliary electrode and/or a reference electrode. Optionally, the auxiliary electrode is a counter electrode. The sensor is configured to detect changes in the electrochemical activity of the at least one redox species at the working electrode using a voltammetric technique.

A sensor using the electrode described above as a working electrode can perform detection of the analyte without the need for additional chemicals. The use of antibodies or fragments thereof provides enhanced selectivity.

Optionally, the sensor is configured to detect changes in the electrochemical activity of the at least one redox species at the working electrode by monitoring the electrochemical activity of the at least one redox species at the working electrode over time using a voltammetric technique.

By monitoring the electrochemical activity of the at least one redox species at the working electrode over time, changes in the analyte presence or concentration can be detected. The ability to monitor the electrochemical activity over time is facilitated by use of the electrode described above as the working electrode because it is not necessary to disturb the system by adding additional chemicals.

Optionally, the voltammetric technique is one of square wave voltammetry, differential pulse voltammetry, or alternating current voltammetry.

Advantageously, square wave voltammetry may be used because it is more sensitive compared to other voltammetric techniques due to the absence of background current. Further, this technique can employ faster scan rates, allowing faster detection of analyte presence or concentration.

Optionally, the sensor is configured for monitoring of the electrochemical activity of the at least one redox species at the working electrode in bodily fluid.

As mentioned above, the sensor is particularly suited for detection of analytes in bodily fluid because of the use of antibodies (or fragments thereof), which are resistant to degradation in physiological fluid.

A method of monitoring an analyte concentration with the electrode or sensor described above comprises the steps of: exposing the electrode to a solution; and monitoring the electrochemical activity of the at least one redox species while the electrode is exposed to the solution.

By monitoring the electrochemical activity of the at least one redox species at

the working electrode, changes in the analyte presence or concentration can be detected.

Optionally, the monitoring of the electrochemical activity of the at least one redox species comprises at least two subsequent measurements of the electrochemical activity of the at least one redox species.

Optionally, between the at least two subsequent measurements no addition of chemicals is carried out.

The ability to monitor the electrochemical activity over time is facilitated by use of the electrode described above as the working electrode because it is not necessary to disturb the system by adding additional chemicals, and because the antibodies/fragments thereof have improved resistance to degradation in physiological fluid.

Optionally, the solution is a bodily fluid.

Optionally, the bodily fluid is interstitial fluid (ISF), preferably dermal ISF.

ISF contains many various markers that can be of interest for detection via a sensor as described herein. In one example the ISF is used to detect cardiac related biomarkers. By determining the cardiac related biomarker levels in the ISF, a bodily fluid is used that has the advantage of being easily accessible and allowing a continuous monitoring of cardiac related biomarkers in a simple and reliable way. More advantageously, by its easy accessibility a continuous measurement of corresponding biomarkers in ISF can be carried out by minimal-invasive sensors without the need of actually obtaining a sample of ISF but rather by direct measurement within the ISF in the body without requiring a dedicated sample collection to be performed by a medical doctor or a trained medical assistant.

Embodiments or examples disclosed in the description and/or figures falling outside the scope of the claims are to be understood as examples useful for understanding the present invention.

Other aspects, advantages, and salient features of the invention will become apparent to those skilled in the art from the following detailed description taken in conjunction with the accompanying drawings.

### BRIEF DESCRIPTION OF THE DRAWINGS

FIG. 1 illustrates an example electrode for an electrochemical immunoassay according to certain examples;

FIG. 2 illustrates an example sensor comprising at least one of the electrode, according to certain examples; and

FIG. 3 illustrates a method of monitoring an analyte concentration with the electrode or sensor according to certain examples.

FIGS. 4A-7 relate to experimental data from a specific example of the disclosure:
a. FIGS. 4A and 4B illustrate two sensing protocols;
b. FIG. 5 shows continuous square wave voltammetry measurements for various concentrations of NT-proBNP recorded over a period of 24 hours;
c. FIGS. 6A and 6B plot decreases in peak current height vs. concentration for the protocols shown in FIGS. 4A and 4B, respectively; and
d. FIG. 7 shows the response of the saturated sensor electrode to surrogate interstitial fluid.

### DETAILED DESCRIPTION

The present invention relates to an electrode for an electrochemical immunoassay, a sensor comprising the electrode, and a method of monitoring an analyte concentration with the electrode or the sensor.

The invention provides a new approach for detecting and/or monitoring the binding of analytes to antibodies or fragments thereof.

The following description of examples of the present disclosure, with reference to the accompanying drawings, is provided to assist in a comprehensive understanding of the present invention, as defined by the claims. The description includes various specific details to assist in that understanding but these are to be regarded as merely exemplary. Accordingly, those of ordinary skill in the art will recognize that various changes and modifications of the examples described herein can be made.

The same or similar components may be designated by the same or similar reference numerals, although they may be illustrated in different drawings.

Detailed descriptions of techniques, structures, constructions, functions or processes known in the art may be omitted for clarity and conciseness, and to avoid obscuring the subject matter of the present disclosure.

The terms and words used herein are not limited to the bibliographical or standard meanings, but, are merely used to enable a clear and consistent understanding of the examples disclosed herein.

Throughout the description and claims, the words “comprise”, “contain” and “include”, and variations thereof, for example “comprising”, “containing” and “including”, means “including but not limited to”, and is not intended to (and does not) exclude other features, elements, components, integers, steps, processes, functions, characteristics, and the like.

Throughout the description and claims, the singular form, for example “a”, “an” and “the”, encompasses the plural unless the context otherwise requires. For example, reference to “an object” includes reference to one or more of such objects.

Throughout the description and claims, language in the general form of “X for Y” (where Y is some action, process, function, activity or step and X is some means for carrying out that action, process, function, activity or step) encompasses means X adapted, configured or arranged specifically, but not necessarily exclusively, to do Y.

Features, elements, components, integers, steps, processes, functions, characteristics, and the like, described in conjunction with a particular aspect, embodiment, example or claim are to be understood to be applicable to any other aspect, embodiment, example or claim disclosed herein unless incompatible therewith.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. It is to be understood that this invention is not limited to the particular methodology, protocols, and reagents described, as these may vary, depending upon the context they are used by those of skill in the art.

While the invention has been shown and described with reference to certain examples, it will be understood by those skilled in the art that various changes in form and detail may be made therein without departing from the scope of the invention, as defined by the appended claims.

Certain examples of the present disclosure provide one or more techniques as disclosed in the appended annex to the description. The skilled person will appreciate that any of these techniques may be applied in combination with any of the techniques described above and illustrated in the Figures.

The term “immunoassay” as used herein refers to a biochemical test for detecting the presence and/or concentration of an analyte by use of antibodies (or fragments thereof).

The term “antibody” herein is used in the broadest sense and encompasses various anti-body structures, including but not limited to monoclonal antibodies, polyclonal antibodies, multispecific antibodies (e.g., bispecific antibodies), and antibody fragments and derivatives so long as they exhibit the desired antigen-binding activity. An antibody of the presently disclosed invention thus can be in any of a variety of forms, including a whole immunoglobulin, an antibody fragment such as Fv, Fab, and similar fragments, a single chain antibody which includes the variable domain complementarity determining regions (CDRs), and the like forms, all of which fall under the broad term “antibody”, as used herein.

The term “antibody fragment” as used herein refers to a portion of a full-length antibody, generally the antigen binding or variable region. Examples of antibody fragments include Fab, Fab′, F(ab′)2 and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)2 fragment that has two antigen binding fragments that are capable of cross-linking antigen, and a residual other fragment (which is termed pFc′). Additional fragments can include minibodies, diabodies, linear antibodies, single-chain antibody molecules, and multispecific antibodies formed from antibody fragments (e.g. bis-scFv). As used herein, “functional fragment” with respect to antibodies, refers to Fv, F(ab) and F(ab′)2 fragments. Fragments of the antibodies of the presently disclosed inventive concepts may be as small as about 4, 5, 6, 7, 8, 9, 10, 11, 12, 13, 14, 15, 16, 17, 18, 19, 20, 21, 22, 23, 24, 25, 26, 27, 28, 29, or 30 to 35, to 40, to 45, to 50, to 75, to 100, or to 150 to 200 to 250 (all inclusive) or more amino acids, for example.

Some types of antibody fragments are defined as follows:
Fab is the fragment that contains a monovalent antigen-binding fragment of an antibody molecule. A Fab fragment can be produced by digestion of whole antibody with the enzyme papain to yield an intact light chain and a portion of one heavy chain.
Fab′ is the fragment of an antibody molecule can be obtained by treating whole antibody with pepsin, followed by reduction, to yield an intact light chain and a portion of the heavy chain. Two Fab′ fragments are obtained per antibody molecule.
Fab′ fragments differ from Fab fragments by the addition of a few residues at the carboxyl terminus of the heavy chain CH1 domain including one or more cysteines from the antibody hinge region.
(Fab′)2 is the fragment of an antibody that can be obtained by treating whole antibody with the enzyme pepsin without subsequent reduction. F(ab′)₂ is a dimer of two Fab′ fragments held together by two disulfide bonds.
Fv is the minimum antibody fragment that contains a complete antigen recognition and binding site. This region consists of a dimer of one heavy and one light chain variable domain in a tight, non-covalent association (VH-VL dimer). It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.
A single chain antibody (SCA) is defined herein as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” or “scFv” antibody fragments. Generally, the Fv polypeptide further comprises a polypeptide linker between the VH and VL domains that enables the sFv to form the desired structure for antigen binding.

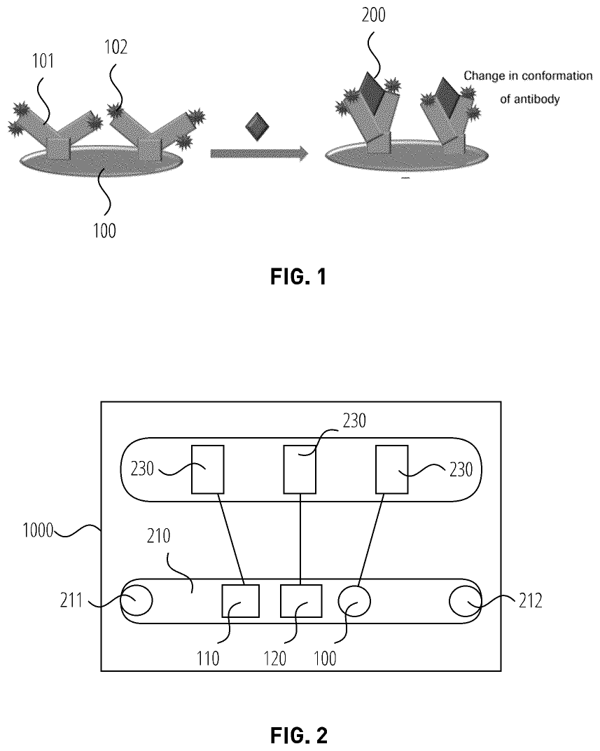

FIG. 1 illustrates an example electrode 100 for an electrochemical immunoassay according to certain examples. The electrode 100 may be functionalized with at least one antibody 101 or fragment thereof, wherein the at least one antibody 101 or fragment thereof is configured to bind to at least one analyte 200. That is, a layer comprising at least one type of antibody 101 (or at least one fragment 101 of at least one type of antibody) may be provided on the electrode surface. The at least one antibody/fragment 101 may be configured to bind to at least one analyte 200, i.e., to at least one substance of interest.

In the case that multiple types of antibody/fragment 101 are used, they may be configured to bind to the same analyte 200, or they may be configured to bind to different analytes 200. For example, the electrode 100 may be functionalized with a first antibody/fragment configured to bind to a first analyte, and a second antibody/fragment configured to bind to a second analyte.

The at least one antibody 101 or fragment thereof is conjugated with at least one redox species 102. This results in an antibody/fragment-redox species conjugate such that electrochemical activity of the at least one redox species 102 is altered when the at least one antibody 101 or fragment thereof binds to the at least one analyte 200. That is, the antibodies/fragments 101 may be “labelled” with redox species molecules 102 that are used as “signal-producing” substances for a determination of the analyte. When the analyte 200 binds to the antibody/fragment 101, this may cause an alteration in the microenvironment of the redox molecule 102 attached to the antibody/fragment 101 and thereby an alteration in the electrochemical activity of the redox species 102 attached to the antibody/fragment 101. The redox activity of a redox molecule 102 is dependent on efficient electron transfer between the redox molecule 102 and the electrode surface. The electron transfer may be perturbed by a change in the microenvironment of the redox molecule 102. Such a change may be caused when the antibody/fragment 101 binds to the analyte 200, for example the change may be caused by the near-presence of the analyte and/or the conformational change of the antibody/fragment 101 upon binding. Although FIG. 1 illustrates this last case (i.e. a conformational change of the antibody/fragment 101 upon binding), it will be appreciated that this is merely an example, and that various mechanisms may cause the measured electrochemical activity of the redox molecules 102 to change when the analyte 200 binds to the antibody/fragment 101.

By measuring the redox activity of the redox species 102, binding of the at least one analyte 200 to the at least one antibody/fragment 101 may be detected, thereby allowing detection of the presence and/or concentration of the at least one analyte 200 when the electrode 100 is exposed to a sample solution (for example, a bodily fluid).

In the case described above in which the electrode 100 may be functionalized with a first antibody/fragment configured to bind a first analyte and a second antibody/fragment configured to bind a second analyte, the first antibody/fragment may be bound to a first type of redox molecule and the second antibody/fragment may be bound to a second type of redox molecule. By selecting the first and second redox molecules such that it is possible to distinguish their electrochemical activities, the electrode 100 may be used to detect changes in the concentration of both the first and second analytes. For example, the first and second redox molecules may be selected to have peaks in redox activity at different voltages or to have different redox potentials, such that the current can be measured at each of the different voltages to measure the electrochemical activity of each redox molecule and thus distinguishably detect signals caused by the first redox molecule or caused by the second redox molecule and thereby distinguishably detect the binding of both the first and second analytes.

In certain examples, the at least one antibody 101 or fragment thereof may be conjugated with the at least one redox species before the electrode 100 is functionalised with the antibodies/fragments 101. That is, the at least one antibody 101 or fragment thereof may be pre-conjugated with the at least one redox species 102.

By pre-conjugating the redox species 102 to the antibodies/fragments 101, difficulties with binding the redox molecules to an already-bound layer of antibodies/fragments 101 may be avoided, and it may be possible to bind the redox molecules to locations on the antibodies/fragments 101 that would not be accessible when the antibodies/fragments 101 are already bound to the electrode surface.

In the case described above in which the electrode 100 may be functionalized with a first antibody/fragment configured to bind a first analyte and a second antibody/fragment configured to bind a second analyte, wherein the first antibody/fragment may be bound to a first type of redox molecule and the second antibody/fragment may be bound to a second type of redox molecule, a pre-conjugation of the different redox species to the different antibodies is advantageous to provide a distinct and definite correlation between signal (caused by the first type of redox molecule or the second type of redox molecule) and analyte (determined by the first antibody/fragment or the second antibody/fragment). That is, by pre-conjugating the different redox species to the different antibodies, undesired conjugation of the first type of redox molecule to the second antibody/fragment (and vice versa) may be avoided.

In certain examples, the at least one antibody 101 or fragment thereof may be conjugated with the at least one redox species 102 via covalent bonding. That is, a covalent bond may be formed between the antibody/fragment 101 and the redox molecule or molecules 102.

In certain examples, the at least one antibody 101 or fragment thereof may be directly attached to the electrode surface (for example may be bound to the electrode via covalent bonding), or may be attached via a linker molecule or molecules (for example may be bound to the electrode via a streptavidin-biotin complex). It will be understood that the antibodies/fragments 101 may be bound such that the binding sites of at least some of the antibodies/fragments 101 will be accessible to analytes 200 in solution. For example, the site at which the antibody/fragment 101 is bound to the electrode 100 may be remote from the analyte binding site.

In certain examples, the electrode 100 may be functionalized with at least one antibody fragment 101. The at least one antibody fragment 101 may comprise, for example, a F(ab′)2 fragment, a Fab fragment, a Fab′ fragment, an (sc)Fv-fragment, or a SMIP (small modular immunopharmaceutical). That is, in certain examples, antibody fragments may be used in addition to, or instead of, whole antibodies.

An advantage of using antibody fragments is that the redox species 102 and/or the analyte binding site of the antibody 101 may be closer to the electrode surface than if a complete antibody is used. Consequently, alterations in the redox activity caused by changes in the microenvironment when the analyte 200 binds to the antibody fragment 101 may be more easily and sensitively detected.

Additionally, or alternatively, the distance from the electrode 100 to the redox species 102 and/or the analyte binding site may be reduced by attaching the redox species 102 to the antibody/fragment 101 at a location close to the location where the antibody/fragment 101 is attached to the electrode 100. However, it will be appreciated that these are just examples, and that detection may be made easier by any technique that reduces the distance between the electrode surface and the redox species 102 and/or analyte binding site. For example: the at least one antibody 101 or fragment thereof may be configured such that an analyte binding site of the at least one antibody 101 or fragment thereof is close to the electrode surface when the electrode 100 is functionalized with the at least one antibody 101 or fragment thereof; the at least one antibody 101 or fragment thereof may be conjugated with the at least one redox species 102 such that the at least one redox species 102 is close to the electrode surface when the electrode 100 is functionalized with the at least one antibody 101 or fragment thereof; the at least one antibody 101 or fragment thereof may be bound to the electrode 100 such that an analyte binding site of the at least one antibody 101 or fragment thereof is close to the electrode surface when the electrode 100 is functionalized with the at least one antibody 101 or fragment thereof; and/or the at least one antibody 101 or fragment thereof may be bound to the electrode 100 such that the at least one redox species 102 is close to the electrode surface when the electrode 100 is functionalized with the at least one antibody 101 or fragment thereof.

In the above examples, the redox species may be considered to be close to the electrode surface if the average distance from the redox species to the electrode surface is 15 nm or less. Preferably, the average distance from the redox species to the electrode surface is 7 nm or less, for example by using F′(ab)′2 fragments. More preferably, the average distance from the redox species to the electrode surface is 4 nm or less, for example by using Fab′ fragments.

In certain examples, the at least one redox species 102 may have an oxidation potential of 0.3 V or less (vs. Ag/AgCl at physiological pH, i.e. 7.2-7.3). In certain examples, the at least one redox species 102 may have an oxidation potential of 0.12 V or less (vs. Ag/AgCl at physiological pH, i.e. 7.2-7.3).

An advantage of using a redox species 102 with a low oxidation potential is that detection can be performed at lower potentials to reduce interference from other redox active substances with higher oxidation potentials that may also be present in the sample. That is, using the lower oxidation potential may reduce the probability that other redox species in the sample will undergo a redox reaction and produce signals that would overlap with the signal from the at least one redox species 102 conjugated to the antibody/fragment 101.

In certain examples, the at least one redox species 102 may be Prussian blue, methylene blue, an osmium complex, or any other suitable redox species for which, when bound to the antibody/fragment 101, a change in electrochemical activity is detectable when the antibody/fragment 101 binds to the analyte 200.

For example, the osmium complex may be as described in patent U.S. Pat. No. 7,045,310 B2 or in Quantitative Analysis of Catalysis and Inhibition at Horseradish Peroxidase Monolayers Immobilized on an Electrode Surface, J. Am. Chem. Soc. 2003, 125, 30, 9192-9203.

In certain examples, the at least one analyte 200 may be at least one (poly)peptide or protein. In certain examples, the peptides or proteins may be cardiac related biomarkers, for example cardiac troponins (CTnT and CTnI), creatine kinase (CK), myoglobins, free fatty acid-binding proteins, apolipoproteins, lipoproteins, and/or C-reactive protein (CRP). In certain examples, the cardiac related biomarkers may be natriuretic peptides, for example BNP, NT-proBNP, ANP, and/or proANP.

The term “(poly)peptide” as used herein refers to chains of amino acids linked by peptide bonds with a length of up to 150 amino acids including oligopeptides, dipeptides, tripeptides, and tetrapeptides.

In the context of present invention, the terms “biomarker”, “marker”, “cardiac related biomarker” and “cardiac related (poly)peptide biomarker are used interchangeably and refer to a peptide or polypeptide (=(poly)peptide) within a biological system that is used as an indicator of a biological state of said system. In the art, the term “biomarker” is sometimes also applied to means for the detection of said endogenous substances (e.g. antibodies, nucleic acid probes etc, imaging systems). In the context of present invention, the term “biomarker” shall be only applied for the substance, not for the detection means. Thus, biomarkers can be any kind of (poly)peptide present in a living organism, such as a protein (cell surface receptor, cytosolic protein etc.), polypeptide, peptide, isomeric form thereof, immunologically detectable fragment thereof, which is differentially present in a subject/sample taken from a subject having HF (heart failure) compared with a subject not having HF.

As used herein, the term “BNP-type peptides” comprise pre-proBNP, proBNP, NT-proBNP, and BNP. The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP). Preferably, BNP-type peptides according to the present invention are NT-proBNP, BNP, and variants thereof. BNP is the active hormone and has a shorter half-life than the respective inactive counterpart NT-proBNP. BNP is metabolized in the blood, whereas NT-proBNP circulates in the blood as an intact molecule and as such is eliminated renally. The in-vivo half-life of NT-proBNP is 120 min longer than that of BNP, which is 20 min (Smith 2000, J Endocrinol. 167:239-46.). Preanalytics are more robust with NT-proBNP allowing easy transportation of the sample to a central laboratory (Mueller 2004, Clin Chem Lab Med 42:942-4.). Blood samples can be stored at room temperature for several days or may be mailed or shipped without recovery loss. In contrast, storage of BNP for 48 hours at room temperature or at 4° Celsius leads to a concentration loss of at least 20 % (Mueller loc. cit. ; Wu 2004, Clin Chem 50:867-73.). Therefore, depending on the time-course or properties of interest, either measurement of the active or the inactive forms of the natriuretic peptide can be advantageous. The most preferred natriuretic peptides according to the present invention are NT-proBNP or variants thereof. As briefly discussed above, the human NT-proBNP, as referred to in accordance with the present invention, is a polypeptide comprising, preferably, 76 amino acids in length corresponding to the N-terminal portion of the human NT-proBNP molecule. The structure of the human BNP and NT-proBNP has been described already in detail in the prior art, e.g., WO 02/089657, WO 02/083913 or Bonow loc.cit. Preferably, human NT-proBNP as used herein is human NT-proBNP as disclosed in EP 0 648 228 B1. These prior art documents are herewith incorporated by reference with respect to the specific sequences of NT-proBNP and variants thereof disclosed therein.

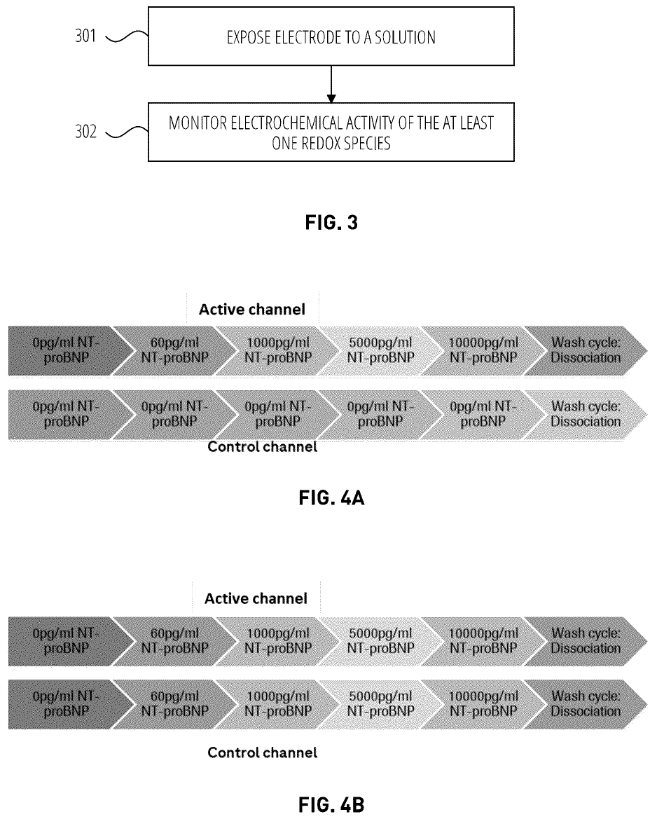

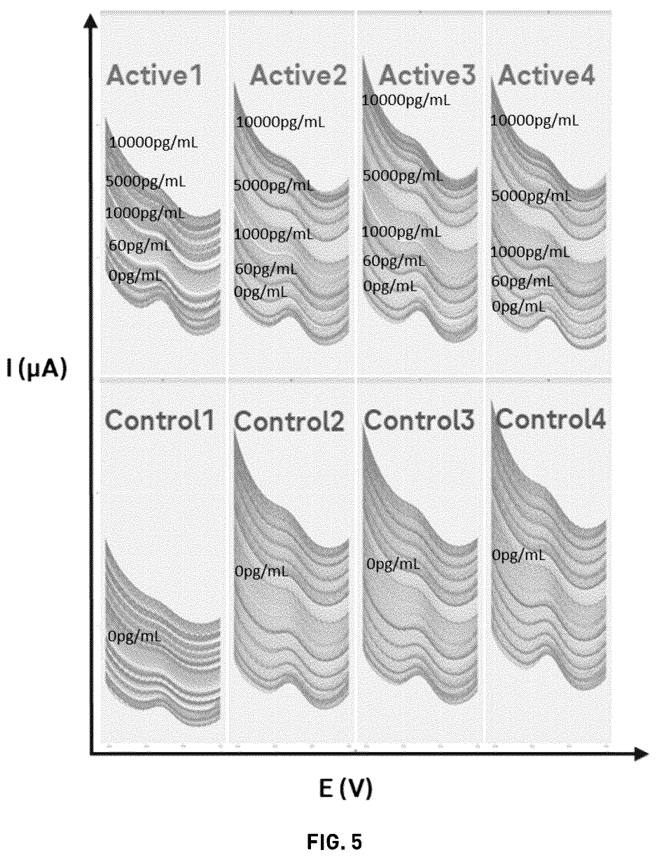

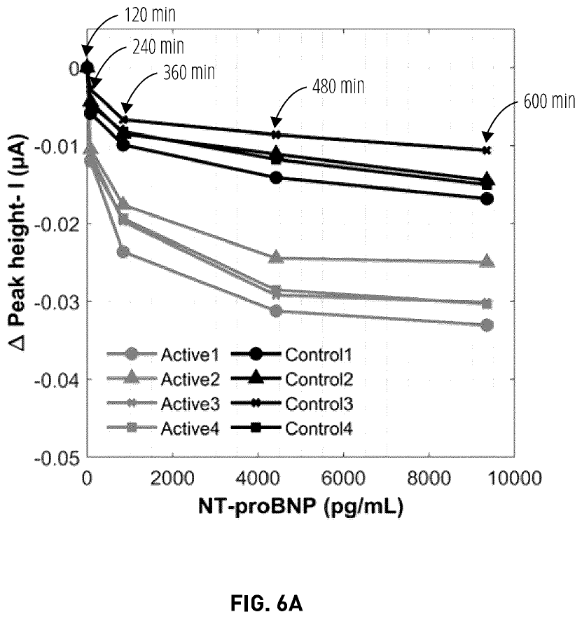

FIG. 2 illustrates an example sensor 1000 comprising at least one of the above-described electrode 100, according to certain examples. The above-described electrode 100 may be at least one working electrode 100 of the sensor 1000. For example, the sensor 1000 may comprise a working electrode 100 functionalized with at least one antibody 101 or fragment thereof configured to bind to at least one analyte 200; wherein the at least one antibody 101 or fragment thereof is conjugated with at least one redox species 102 such that the electrochemical activity of the at least one redox species 102 is altered when the at least one antibody 101 or fragment thereof binds to the at least one analyte 200.

It will be appreciated that all the features of the above-described electrode 100 are applicable to the at least one working electrode 100 of the sensor 1000. That is, any of the example electrodes 100 described above may be used as the working electrode 100 of the sensor 1000.

The sensor 1000 may further comprise an auxiliary electrode 110 and/or a reference electrode 120. That is, in addition to the at least one working electrode 100, the sensor may comprise at least one auxiliary (or counter) electrode 110 and/or at least one reference electrode 120.

Examples of possible working electrodes, auxiliary (or counter) electrodes, and reference electrodes (including possible materials etc.) may be found in patent application EP 3 838 147 A1. However, it will be appreciated that any suitable working electrodes, auxiliary (or counter) electrodes, and reference electrodes may be used.

The sensor 1000 may be configured to detect changes in the electrochemical activity of the at least one redox species 102 at the at least one working electrode 100 using a voltametric technique. The electrochemical activity of the at least one redox species 102 may be detected by measuring the current at the working electrode 100 as the potential between the working electrode 100 and the reference electrode 120 is varied.

The sensor 1000 may further comprise a fluidic channel 210 comprising an inlet 211 and an outlet 212. The at least one working electrode 100, at least one auxiliary electrode 110, and at least one reference electrode 120 may be located within the fluidic channel, such that the electrodes are exposed to a sample solution when the sample solution is present in the fluidic channel.

The sensor 1000 may further comprise a plurality of conductive connecting pads 230 that are each electrically connected to one of the at least one working electrode 100, at least one auxiliary electrode 110, and at least one reference electrode 120. The connecting pads may enable electrical connection to be made between the electrodes of sensor 1000 and an exterior device such as a potentiostat and/or computing device. For example, a first connecting pad may be electrically connected to a working electrode 100, a second connecting pad may be electrically connected to an auxiliary electrode 110, and a third connecting pad may be electrically connected to a reference electrode 120. The connecting pads may be disposed on an area of the sensor outside the fluidic channel, such that the connecting pads do not make contact with the sample solution.

In certain examples, the sensor 1000 may be configured to detect changes in the electrochemical activity of the at least one redox species 102 at the working electrode 100 by monitoring the electrochemical activity of the at least one redox species 102 at the working electrode 100 over time using a voltammetric technique. For example, a first measurement of the electrochemical activity of the at least one redox species 102 may be performed at a first time, a second measurement of the electrochemical activity of the at least one redox species 102 may be performed at a second time, and the first and second measurements may be compared to detect changes in the electrochemical activity. Such a change may be indicative of a change of concentration of the at least one analyte 200 over time.

In certain examples, comparing the first and second (and subsequent) measurements may comprise comparing peak current values of the measurements, or comparing current values of the measurements at a particular voltage.

In certain examples, between the subsequent measurements no addition of chemicals is carried out, in particular no addition of external solutions or reagents to at least partially remove bound analyte molecules from the antibody or the fragment thereof. This means that no additional step like a washing step, or a step for dissociating the analyte from the antibody or fragment thereof is carried out. In this context a skilled person is aware what is meant by “chemicals”. For example, washing buffers or any other reagents that would be considered for washing the electrode as it would for example be necessary in an ELISA or sandwich immunoassay.

In a continuous measurement as described herein the analyte dissociates from the antibody or fragment thereof without any external influence, i.e. without triggering a (rapid) change of the environmental conditions resulting in the dissociation of the analyte from the antibody or fragment thereof. The dissociation rather occurs due to naturally occurring changes of the environmental conditions (passive regeneration) that are not triggered by the addition of external chemicals/reagents. This can, for example, be due to changes in equilibria based on concentration changes of components contained in the sample, changes in the concentration of the analyte in the sample etc. For example, the short half-life of NT-proBNP in body can rapidly change its concentration in dermal ISF modulating the equilibria, and thereby triggering the dissociation of NT-proBNP from the antibody fragment. In this context a skilled person is aware that the binding properties between the analyte and an antibody or a fragment thereof are significantly determined by the kinetic properties of the antibody or the fragment thereof, like e.g. its association rate constant kₐ, its dissociation rate constant k_{d}, its dissociation constant K_{D} or its halftime of complex dissociation t_{1/2}(diss). For example, for NT-proBNP-binding antibodies such kinetic properties are disclosed e.g. in WO 2018/146321, including the methods for determining such kinetic properties. In this context a person skilled in the art is aware that for such continuous measurement applications a balanced ratio between the respective association rates and dissociation rates is preferred that allows both a reliable and sensitive binding of the analyte as well as an adequate and sufficient dissociation of bound analyte molecules that is important for performing subsequent measurements without any washing steps. This allows to use the sensor disclosed herein for continuous in-situ measurements, for example as wearable or implantable sensors.

By repeatedly measuring the electrochemical activity of the at least one redox species 102 over time it may be possible to perform continuous monitoring of the analyte 200 concentration. That is, in certain examples the sensor 1000 may be configured to continuously monitor the analyte 200 concentration by performing a plurality of subsequent measurements of the electrochemical activity of the at least one redox species 102.

Several characteristics of the electrode and/or sensor as disclosed herein play a role when a monitoring over time should be carried out. The following factors can have an impact on the suitability for long-term monitoring:
Choice of redox species
Choice of conjugation between antibody or fragment thereof and the redox species
Choice of conjugation between antibody or fragment thereof and the electrode
Size and/or stability of the antibody or fragment thereof
Distance between the antibody or fragment thereof and the electrode
Specificity and/or affinity of the antibody or fragment thereof
Type and/or size of analyte

### Surrounding Environmental Conditions

These factors alone or in combination have an influence on the suitability of an electrode and/or sensor for long-term monitoring, in particular without requiring the addition of chemicals between subsequent measurements.

When at least two or preferably more factors are applied in combination, a synergistic effect enabling a long-term monitoring can be achieved.

In one example the antibody or fragment thereof of the electrode as described herein may be bound to the electrode via covalent bonding and may be conjugated via covalent bonding to a redox species.

In one example the antibody or fragment thereof of the electrode as described herein may be a F(ab′)2 fragment and may be bound to the electrode via covalent bonding and may be conjugated via covalent bonding to a redox species.

In one example the antibody or fragment thereof of the electrode as described herein may be a F(ab′)2 fragment and may be bound to the electrode via covalent bonding and may be conjugated via covalent bonding to a redox species, wherein the analyte is NT-proBNP.

In one example the antibody or fragment thereof of the electrode as described herein may be a F(ab′)2 fragment and may be bound to the electrode via covalent bonding and may be conjugated via covalent bonding to a redox species, wherein the analyte is NT-proBNP measured in ISF.

In a preferred embodiment, it is advantageous to use an osmium complex as a redox species, in particular to conjugate the antibody or a fragment thereof to an osmium complex.

In one example the antibody or fragment thereof of the electrode as described herein may be conjugated to an osmium complex as redox species, preferably bis(1,1′-dimethyl-biimidazolyl)-2-(1-heptyl-imidazol-)6-methyl-pyridin-Os3+.

In one example the antibody or fragment thereof of the electrode as described herein may be conjugated via covalent bonding to an osmium complex as redox species, preferably bis(1,1′-dimethyl-biimidazolyl)-2-(1-heptyl-imidazol-)6-methyl-pyridin-Os3+.

In one example the antibody or fragment thereof of the electrode as described herein may be bound to the electrode via covalent bonding to the electrode and may be conjugated via covalent bonding to an osmium complex as redox species, preferably bis(1,1′-dimethyl-biimidazolyl)-2-(1-heptyl-imidazol-)6-methyl-pyridin-Os3+.

In one example the antibody or fragment thereof of the electrode as described herein may be a F(ab′)2 fragment and may be bound to the electrode via covalent bonding and may be conjugated via covalent bonding to an osmium complex as redox species, preferably bis(1,1′-dimethyl-biimidazolyl)-2-(1-heptyl-imidazol-)6-methyl-pyridin-Os3+.

In one example the antibody or fragment thereof of the electrode as described herein may be a F(ab′)2 fragment and may be bound to the electrode via covalent bonding and may be conjugated via covalent bonding to an osmium complex as redox species, preferably bis(1,1′-dimethyl-biimidazolyl)-2-(1-heptyl-imidazol-)6-methyl-pyridin-Os3+, wherein the analyte is NT-proBNP.

In one example the antibody or fragment thereof of the electrode as described herein may be a F(ab′)2 fragment and may be bound to the electrode via covalent bonding and may be conjugated via covalent bonding to an osmium complex as redox species, preferably bis(1,1′-dimethyl-biimidazolyl)-2-(1-heptyl-imidazol-)6-methyl-pyridin-Os3+, wherein the analyte is NT-proBNP measured in ISF.

In certain examples, the voltammetric technique may be, for example, square wave voltammetry, differential pulse voltammetry, or alternating current voltammetry. However, it will be appreciated that these are just examples, and that any suitable voltammetric technique could be used.

In certain examples, the sensor 1000 may be configured for monitoring of the electrochemical activity of the at least one redox species 102 at the working electrode 100 in bodily fluid. That is, the sensor 1000 may be configured to detect changes of the electrochemical activity of the at least one redox species 102 when the working electrode 100 is exposed to bodily fluid.

In certain examples, the bodily fluid may be interstitial fluid, and the at least one analyte 200 may be at least one analyte 200 that may be present in interstitial fluid.

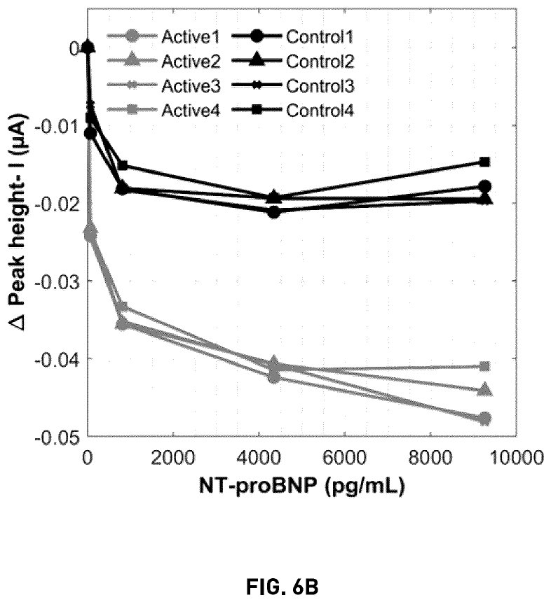

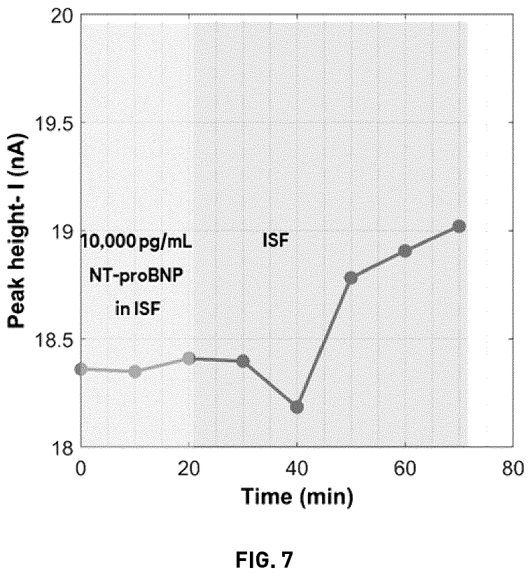

FIG. 3 illustrates a method of monitoring an analyte concentration with the above-described electrode or sensor according to certain examples.

That is, in certain examples the method may be performed using a working electrode 100 functionalized with at least one antibody 101 or fragment thereof configured to bind to at least one analyte 200; wherein the at least one antibody 101 or fragment thereof is conjugated with at least one redox species 102 such that the electrochemical activity of the at least one redox species 102 is altered when the at least one antibody 101 or fragment thereof binds to the at least one analyte 200. Also, in certain examples, the method may be performed using a sensor 1000 comprising the electrode 100 as a working electrode 100, wherein the sensor further comprises an auxiliary electrode 110 and/or a reference electrode 120, and wherein the sensor 1000 is configured to detect changes in the electrochemical activity of the at least one redox species 102 at the working electrode 100 using a voltametric technique.

The method may comprise a first step 301 of exposing the electrode 100 (which may be the working electrode 100 of the sensor 1000) to a solution, and a second step 302 of monitoring the electrochemical activity of the at least one redox species 102 while the electrode 100 is exposed to the solution.

It will be appreciated that all the features of the above-described electrode 100 and sensor 1000 are applicable to the method 300. That is, the method may be performed using any of the example electrodes 100 and sensors 1000 described above.

As described above, in certain examples the solution may be a bodily fluid. In certain examples, the bodily fluid may be interstitial fluid. Further examples of bodily fluid include but are not limited to blood, serum, plasma, synovial fluid, interstitial fluid, capillary blood, peritoneal fluid, menstrual fluid, urine, saliva, and lymphatic fluid.

FIGS. 4A-7 relate to experimental data from a specific example of the disclosure. To produce the immunoassay, working electrodes were functionalized with F(ab′)2 antibody fragments that specifically bind to the analyte NT-pro-BNP. Specifically, gold working electrodes were functionalized with carboxy-PEG-thiol. Then redox labelled antibody fragments were conjugated with the carboxy group using EDC/NHS chemistry. Open sites/pin holes on the electrode surface were blocked with BSA based buffer. The redox molecules used to label the antibody fragments were bis(1,1′-dimethyl-biimidazolyl)-2-(1-heptyl-imidazol-)6-methyl-pyridin-Os3+. Electrochemical activity was measured using square wave voltammetry.

FIGS. 4A and 4B illustrate two sensing protocols. The active sensor channel in each figure comprised four working electrodes (Active1, Active2, Active3, Active 4) with osmium complex labeled antibody fragments (Os-NT-proBNP-F(ab′)2) that specifically bind to NT-proBNP. The concentration of NT-proBNP used in the active channel in each protocol was 0, 60, 1000, 5000, and 10,000 pg/mL in surrogate interstitial fluid solution. For each concentration stage, the sensor was incubated for 2 hours before measurement. In FIG. 4A, the control sensor channel comprised four working electrodes (Control1, Control2, Control3, Control 4) which were also modified with Os-NT-proBNP-F(ab′)2, and the concentration of NT-proBNP used in the control channel was 0 pg/mL in surrogate interstitial fluid at each stage. That is, the concentration shown on the x-axis of FIG. 6A is correct only for the active sensor channel, since the concentration was 0 pg/ml for each measurement of the control sensor channel. For each concentration stage in both the control and active sensor channels, the sensor was incubated for 2 hours before measurement. That is, as shown on FIG. 6A, the measurements were performed at 120 minutes, 240 minutes, 360 minutes, 480 minutes, and 600 minutes.

In FIG. 4B the experiment of FIG. 4A was repeated but with a different control sensor channel. The working electrodes of the control sensor channel in the experiment of FIG. 4B were modified with non-specific antibodies also labelled with osmium (Os-MAK33). That is, the antibodies were not specific to NT-proBNP. The concentration of NT-proBNP used in the control channel was 0, 60, 1000, 5000, and 10,000 pg/mL in surrogate interstitial fluid solution (i.e. the same as the active channel). For each concentration stage, the sensor was incubated for 2 hours before measurement.

FIG. 5 shows continuous square wave voltammetry measurements for an experiment as described in relation to FIG. 4A. The various concentrations of NT-proBNP were recorded over a period of 24 hours (in the control channel, the concentration was 0 pg/ml for all measurements). Square wave voltammetry was recorded after every 5 minutes, with a voltage range of- 0.5 to 0 V vs. Ag/AgCl, with frequency 500 Hz. For each concentration, the sample was allowed to flow for 30 minutes and then the flow was stopped for 90 minutes, giving a total of 120 minutes exposure for each concentration.

FIGS. 6A and 6B plot decreases in peak current height (derived from the corresponding square wave voltammetry measurement for each last measurement point of a given concentration, i.e. the change in peak height after 120 minutes incubation for each concentration) vs. concentration for the protocols shown in FIGS. 4A and 4B, respectively. In both figures, the active (grey) and control (black) sensor can be well differentiated. The aim of the experiment in FIG. 6A is to show that for the electrodes modified with specific antibodies, peak height decreases to a greater extent only in presence of NT-proBNP. In absence of NT-proBNP (i.e. in the control channel), a decrease in peak height in a similar range is not observed. The decrease in peak height over time that is also visible for the control channel can be related to unspecific changes in the sensor characteristics over time or to interactions of the electrodes with other ingredients of the ISF surrogate solution that take place in all channels. But in all cases, the decreases in peak height of the active channel are greater that the respective decreases in peak height of the control channel, indicating that the changes in the decrease in peak height are correlated to the concentration of the analyte NT-proBNP in the ISF surrogate solution. The aim of the experiment in FIG. 6B is to show that for the electrodes modified with specific antibodies (i.e. the active channel), peak height decreases to a greater extent in presence of NT-proBNP. In the electrodes modified with non-specific antibodies (i.e. the control channel), similar decrease in peak height is not observed. Thus, FIGS. 6A and 6B collectively show that the decrease in peak height of the square wave voltammetry is due to specific binding of the specific antibody (i.e. Os-NT-proBNP-F(ab′)2) and NT-proBNP,, which changes the microenvironment of the antibody, thereby affecting the electron transfer from osmium label to sensor electrode.

Note that the concentration values plotted in FIGS. 6a and 6b are not the theoretical (i.e. intended) values according to the protocols shown in FIGS. 4a and 4b, but are experimentally determined (i.e. actual) concentration values using the commercially available Elecsys test method for the analyte.

FIG. 7 shows the response of the sensor working electrode (i.e. a working electrode modified with Os-NT-proBNP-F(ab′) 2) saturated with 10,000 pg/mL of NT-proBNP to surrogate interstitial fluid. According to FIG. 7, after 20 minutes the sample solution was exchanged to pure ISF surrogate solution with 0 pg/mL of NT-proBNP. Here, owing to the dissociation of NT-proBNP from the antibody, the peak current increases again over time. This passive regeneration caused by the dissociation of the bound analyte, could facilitate continuous monitoring of biomarkers without the use of any additional chemical or reagent.

1. An electrode for an electrochemical immunoassay;
wherein the electrode is functionalized with at least one antibody or fragment thereof configured to bind to at least one analyte; and
wherein the at least one antibody or fragment thereof is conjugated with at least one redox species such that the electrochemical activity of the at least one redox species is altered when the at least one antibody or fragment thereof binds to the at least one analyte.

2. The electrode of claim 1, wherein the at least one antibody or fragment thereof is conjugated with the at least one redox species via covalent bonding.

3. The electrode of claim 1, wherein the at least one antibody or fragment thereof is bound to the electrode via covalent bonding;
or wherein the at least one antibody or fragment thereof is bound to the electrode via a streptavidin-biotin complex.

4. The electrode of claim 1, wherein the electrode is functionalized with at least one antibody fragment comprising a F(ab′)2 fragment, a Fab fragment, a Fab′ fragment, an (sc)Fv-fragment, or a SMIP (small modular immunopharmaceutical).

5. The electrode of claim 1, wherein at least one redox species has an oxidation potential of 0.3 V or less.

6. The electrode of claim 1, wherein at least one redox species is one of Prussian blue, methylene blue, or an osmium complex.

7. The electrode of claim 1, wherein at least one analyte is a peptide or protein.

8. The electrode of claim 1, wherein at least one analyte is a natriuretic peptide, preferably NT-proBNP.

9. A sensor comprising the electrode of claim 1, wherein the electrode is a working electrode of the sensor;
wherein the sensor further comprises an auxiliary electrode and/or a reference electrode; and
wherein the sensor is configured to detect changes in the electrochemical activity of the at least one redox species at the working electrode using a voltametric technique.

10. The sensor of claim 9, wherein the sensor is configured to detect changes in the electrochemical activity of the at least one redox species at the working electrode by monitoring the electrochemical activity of the at least one redox species at the working electrode over time using a voltametric technique.

11. The sensor of claim 9, wherein the voltametric technique is one of square wave voltammetry, differential pulse voltammetry, or alternating current voltammetry.

12. The sensor of any of claims 9, wherein the sensor is configured for monitoring of the electrochemical activity of the at least one redox species at the working electrode in bodily fluid.

13. A method of monitoring an analyte concentration with the electrode or sensor of claim 1, the method comprising:
exposing the electrode to a solution; and
monitoring the electrochemical activity of the at least one redox species while the electrode is exposed to the solution.

14. The method according to claim 13, wherein the monitoring of the electrochemical activity of the at least one redox species comprises at least two subsequent measurements of the electrochemical activity of the at least one redox species.

15. The method according to claim 13, wherein between the at least two subsequent measurements no addition of chemicals is carried out.

16. The method of claim 13, wherein the solution is a bodily fluid.

17. The method of claim 16, wherein the bodily fluid is interstitial fluid.

18. The method of claim 17, wherein the bodily fluid is dermal interstitial fluid.

19. The method of claim 13, wherein
a) the monitoring of the analyte concentration is carried out with an electrode functionalized with at least one antibody or fragment thereof configured to bind to at least one analyte; and wherein the at least one antibody or fragment thereof is conjugated with at least one redox species such that the electrochemical activity of the at least one redox species is altered when the at least one antibody or fragment thereof binds to the at least one analyte; or
b) wherein the monitoring of the analyte concentration is carried out with a sensor, wherein the electrode of a) is a working electrode of the sensor; wherein the sensor further comprises an auxiliary electrode and/or a reference electrode; and wherein the sensor is configured to detect changes in the electrochemical activity of the at least one redox species at the working electrode using a voltametric technique.
